(19) United States
(12) Patent Application Publication
(10) Pub. No.: US 2018/0064892 A1
(43) Pub. Date: Mar. 8, 2018

(54) INTRA-NASAL CLIP DEVICE

(71) Applicant: DANIEL D. NELSON, Star, ID (US)

(72) Inventors: DANIEL D. NELSON, Star, ID (US); John Warren, Nampa, ID (US)

(73) Assignee: DANIEL D. NELSON, Star, ID (US)

(21) Appl. No.: 15/674,892

(22) Filed: Aug. 11, 2017

Related U.S. Application Data
(60) Provisional application No. 62/383,458, filed on Sep. 4, 2016.

(51) Int. Cl. A61M 15/08; A62B 9/06

(52) U.S. Cl. CPC A61M 15/08; A62B 9/06; A61M 15/085

(57) ABSTRACT

An intra-nasal clip device may include a retainer having a first arm and a second arm. A first chamber housing may be coupled to the first arm, and the first chamber housing may include a first chamber and a first contact surface. Preferably, the first chamber and the first contact surface may be positioned on opposing sides of the first chamber housing. A second chamber housing may be coupled to the second arm, and the second chamber housing may include a second chamber and a second contact surface. Preferably, the second chamber and second contact surface may be positioned on opposing sides of the second chamber housing. The first contact surface and second contact surface may be orientated towards each other and configured to contact and grip portions of the nose so that a chamber, optionally containing an aromatic compound, may be positioned within a nostril of the nose.

### CROSS REFERENCE TO RELATED APPLICATIONS

This application claims priority to and the benefit of the filing date of U.S. Provisional Application No. 62/383,458, filed on Sep. 4, 2016, entitled “INTRA-NASAL CLIP DEVICE”, which is hereby incorporated by reference in its entirety.

### FIELD OF THE INVENTION

This patent specification relates to the field of devices configured for the nasal administration of compounds. More specifically, this patent specification relates to an intra-nasal clip that facilitates the transmission and dispensing of aromas, vapors, medicines, drugs, fragrances, or other compounds directly into the nasal cavity.

### BACKGROUND

Aromatherapy is the art and science of utilizing naturally extracted aromatic essences from plants, known as essential oils, to provide a variety of therapeutic benefits including the sense of well-being, relief of motion sickness, nasal congestion, many other known benefits to the body and mind.

Common techniques for using aromatherapy include application of essential oils to the skin through massage, adding essential oils to bath water, and through inhalation of volatile essential oil vapors.

It is widely accepted that essential oils that are inhaled directly into the lungs offer increased psychological and physical benefits. Not only does the aroma of the natural essential oil stimulate the brain to trigger a reaction, but when inhaled into the lungs or absorbed through the skin, the naturally occurring chemicals can supply therapeutic benefit.

One current method of providing a means of inhaling essential oils is through a variety of electronic vaporizers and natural evaporators that emit the aromatherapy aroma into the surrounding air, where it can be inhaled. This means of vaporization does not create a concentrated aroma, and thus has limited benefit. In addition to not providing a concentrated essential oil aroma, the common vaporizers and evaporators are not considered portable or usable in large rooms or outdoors.

One means of portable aromatherapy is to apply an essential oil soaked adhesive backed patch of absorbent material to the area of the face near the nasal passages. The evaporation of volatile essential oils occurs near the nasal inlet, creating a more concentrated aroma than the room type vaporizers. However, this method can be an ineffective means of essential oil aroma distribution if the user is moving or is an area where wind could blow the aroma away from the nasal passages.

Therefore, a need exists for novel devices that will distribute essential oil aroma directly into the nasal cavity (Intra-nasal). Intra-nasal distribution will provide the most direct and concentrated vapors into the lungs without losing aroma concentration because of external forces such as wind and motion. A further need exists for novel devices that facilitate the transmission and dispensing of aromas, vapors, medicines, drugs, fragrances, or other compounds directly into the nasal cavity.

### BRIEF SUMMARY OF THE INVENTION

An intra-nasal clip device is provided. The device may be used to enable aromatic compounds, which may be contained in the device, to be insufflated through the nose. In some embodiments, the device may include a retainer having a first arm and a second arm. A first chamber housing may be coupled to the first arm, and the first chamber housing may include a first chamber and a first contact surface. Preferably, the first chamber and the first contact surface may be positioned on opposing sides of the first chamber housing. A second chamber housing may be coupled to the second arm, and the second chamber housing may include a second chamber and a second contact surface. Preferably, the second chamber and second contact surface may be positioned on opposing sides of the second chamber housing. The first contact surface and second contact surface may be orientated towards each other and configured to contact and grip portions of the nose so that a chamber may be positioned within a nostril of the nose.

In further embodiments, the device may be for use with a nose having a septum which separates a first nostril from a second nostril. The device may include a retainer having a first arm and a second arm. A first chamber housing may be coupled to the first arm, and the first chamber housing may include a first chamber and a first contact surface. The first chamber and first contact surface may be positioned on opposing sides of the first chamber housing, and the first contact surface may be configured to contact the septum within the first nostril. A second chamber housing may be coupled to the second arm, and the second chamber housing may include a second chamber and a second contact surface. The second chamber and second contact surface may be positioned on opposing sides of the second chamber housing, and the first contact surface and second contact surface may be orientated towards each other, and the second contact surface may be configured to contact the septum within the second nostril. The first contact surface and second contact surface may be orientated towards each other and configured to contact and grip portions of the nose so that a chamber may be positioned within a nostril of the nose.

In still further embodiments, the device may comprise a bridge which may be used to couple the first arm to the second arm. The bridge may allow a first contact surface and a second contact surface to contact and be frictionally retained on a portion of the septum posterior to the columella while preventing the first arm and second arm from squeezing or exerting uncomfortable pressure on the columella.

In still further embodiments, the device may comprise one or more turbulators. A turbulator may comprise or form a protrusion which extends above a chamber to direct a flow of air across the chamber housings to eddy or otherwise become turbulent over a chamber thereby increasing or promoting the dispersion of an aromatic compound from a chamber into the air flow.

### BRIEF DESCRIPTION OF THE DRAWINGS

Some embodiments of the present invention are illustrated as an example and are not limited by the figures of the accompanying drawings, in which like references may indicate similar elements and in which:

FIG. 1 depicts a perspective view of an example of an intra-nasal clip device according to various embodiments described herein.

FIG. 2 illustrates a plan view of an example of an intra-nasal clip device according to various embodiments described herein.

FIG. 3 shows an elevation view of an example of an intra-nasal clip device according to various embodiments described herein.

FIG. 4 depicts a side view of an example of an intra-nasal clip device according to various embodiments described herein.

FIG. 5 illustrates a sectional, through line 5-5 shown in FIG. 4, elevation view of an example of an intra-nasal clip device according to various embodiments described herein.

FIG. 6 shows a side view of an example of an intra-nasal clip device containing an aromatic compound according to various embodiments described herein.

FIG. 7 depicts a sectional, through line 7-7 shown in FIG. 6, elevation view of an example of an intra-nasal clip device containing an aromatic compound according to various embodiments described herein.

FIG. 8 illustrates a plan view of a further example of an intra-nasal clip device according to various embodiments described herein.

FIG. 9 shows a plan view of an alternative example of an intra-nasal clip device according to various embodiments described herein.

FIG. 10 depicts a perspective view of an example of an intra-nasal clip device positioned within portions of a nose according to various embodiments described herein.

FIG. 11 illustrates a perspective sectional view of still another example of an intra-nasal clip device positioned within portions of a nose according to various embodiments described herein.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

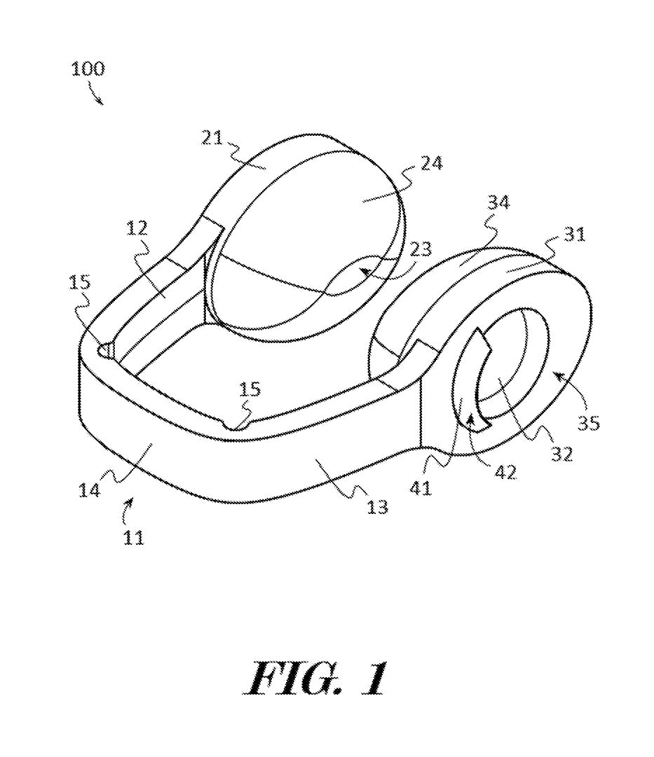

For purposes of description herein, the terms “upper”, “lower”, “left”, “right”, “rear”, “front”, “side”, “vertical”, “horizontal”, and derivatives thereof shall relate to the invention as oriented in FIG. 1. However, one will understand that the invention may assume various alternative orientations and step sequences, except where expressly specified to the contrary. Therefore, the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Although the terms “first”, “second”, etc. are used herein to describe various elements, these elements should not be limited by these terms. These terms are only used to distinguish one element from another element. For example, the first element may be designated as the second element, and the second element may be likewise designated as the first element without departing from the scope of the invention.

As used in this application, the term “about” or “approximately” refers to a range of values within plus or minus 10% of the specified number. Additionally, as used in this application, the term “substantially” means that the actual value is within about 10% of the actual desired value, particularly within about 5% of the actual desired value and especially within about 1% of the actual desired value of any variable, element or limit set forth herein.

New devices configured for the nasal administration of compounds are discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention, and is not intended to limit the invention to the specific embodiments illustrated by the figures or description below.

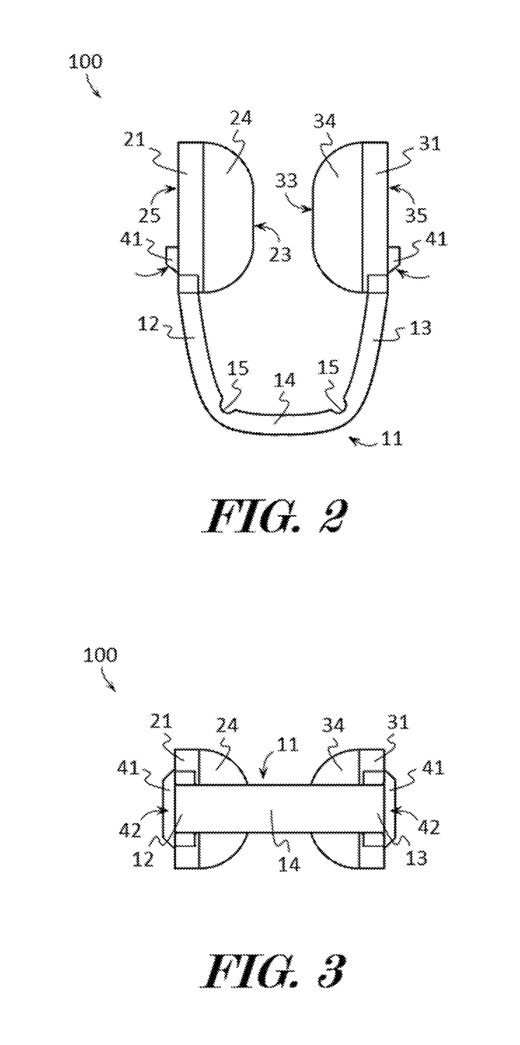

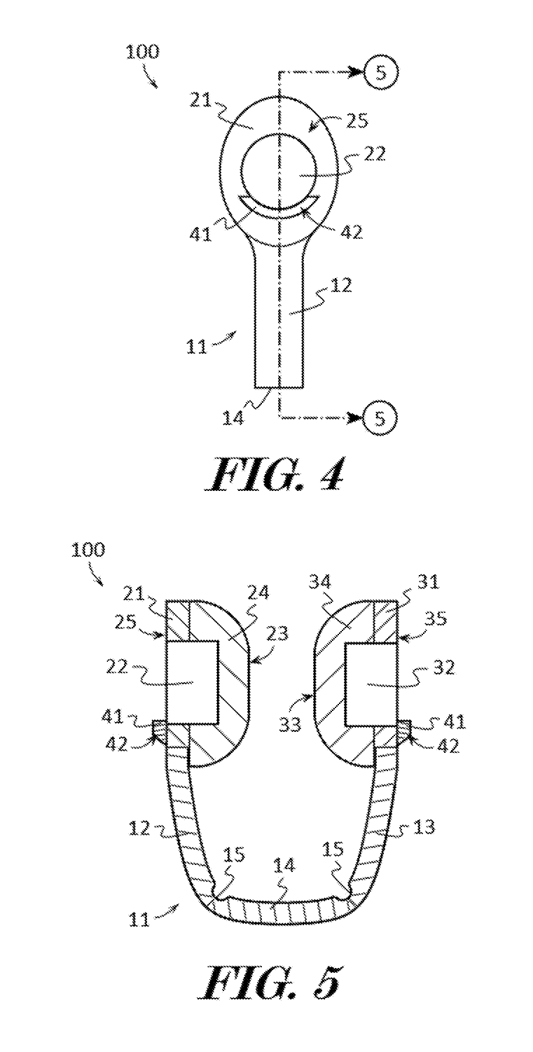

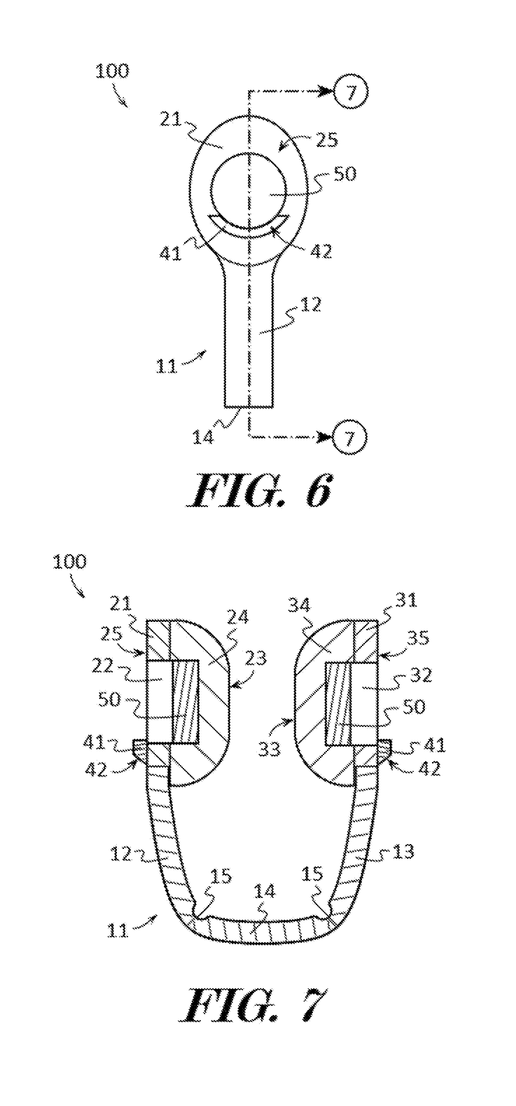

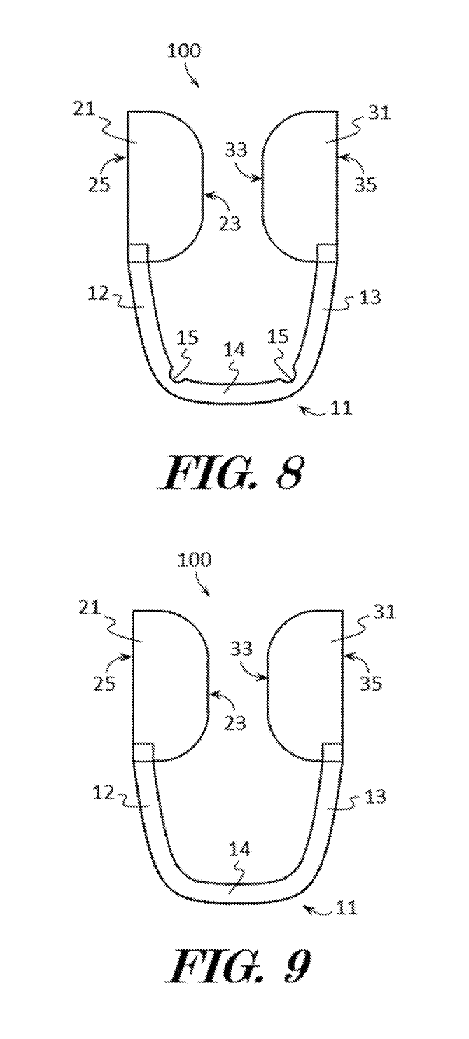

The present invention will now be described by example and through referencing the appended figures representing preferred and alternative embodiments. FIGS. 1-11 illustrate examples of an intra-nasal clip device (“the device”) 100 according to various embodiments. In some embodiments, the device 100 may comprise a retainer 11 having a first arm 12 and a second arm 13. A first chamber housing 21 may be coupled to the first arm 12, and the first chamber housing 21 may comprise a first chamber 22 and a first contact surface 23. Preferably, the first chamber 22 and the first contact surface 23 may be positioned on opposing sides of the first chamber housing 21. A second chamber housing 31 may be coupled to the second arm 13, and the second chamber housing 31 may comprise a second chamber 32 and a second contact surface 33. Preferably, the second chamber 32 and second contact surface 33 may be positioned on opposing sides of the second chamber housing 31. The first contact surface 23 and second contact surface 33 may be orientated towards each other and configured to contact and grip portions of the nose 200 so that a chamber 22, 32, may be positioned within a nostril 201, 202, of the nose 200.

The device 100 may comprise one or more chamber housings 21, 31. In preferred embodiments, the device 100 may comprise a first chamber housing 21 and a second chamber housing 31. Each chamber housing 21, 31, may comprise a chamber 22, 32, and a contact surface 23, 33. In some embodiments, a chamber 22, 32, and a contact surface 23, 33 of a chamber housing 21, 31, may be disposed or positioned on opposite sides of the respective chamber housing 21, 31.

Each chamber housing 21, 31, may comprise one or more contact surfaces 23, 33. In some embodiments, a first chamber housing 21 may comprise one or more first contact surfaces 23 and/or a second chamber housing 31 may comprise one or more second contact surfaces 33. Generally, the contact surfaces 23, 33, form the point of contact between the device 100 and portions of the nose 200 to which the device 100 may be engaged. For example, portions of the nose 200 of a user may be positioned between and in contact with a first contact surface 23 of a first chamber 21 and a second contact surface 33 of a second chamber 31 to enable the chamber housings 21, 31, to grip or frictionally retain the portions of the nose 200 disposed between the contact surfaces 23, 33.

In some embodiments, a first chamber housing 21 may comprise a flexible material which may form all or portions of a first contact surface 23 and/or a second chamber housing 31 may comprise a flexible material which may form all or portions of a second contact surface 33. In preferred embodiments, the first chamber housing 21 may comprise a first cushion 24 of flexible material forming a first contact surface 23 and the second chamber housing 31 may comprise a second cushion 34 of flexible material forming a second contact surface 33 with the cushions 24, 34, functioning to increase user comfort when portions of the nose 200 of a user are gripped or frictionally retained between a first chamber housing 21 and a second chamber housing 31. In some embodiments, a flexible material of a contact surface 23, 33, may comprise silicone rubber, silicone foams, rubber foams, urethane foams including plastic foams, neoprene foam, latex foam rubber, polyurethane foam rubber, or elastomer materials such as elastic plastics, elastic silicone, elastic rubbers, or any other suitable elastomer or flexible material suitable for providing cushioning contact to the skin on the outside and inside of the nose including combinations of materials.

The device 100 may comprise one or more chambers 22, 32. In preferred embodiments, a first chamber housing 21 may comprise one or more first chambers 22 and/or a second chamber housing 31 may comprise one or more second chambers 32. A first chamber 22 may form a depression, recess, cavity, channel, or the like, which may be suitable for containing a volume of a substance, such as an aromatic compound 50 within a first chamber housing 21. A first chamber 22 may be positioned anywhere on a first chamber housing 21, but preferably a first chamber 22 may be positioned on a side of a first chamber housing 21 that is opposite to a side that a first contact surface 23 may be positioned.

Likewise, a second chamber 22 may form a depression, recess, cavity, channel, or the like, which may be suitable for containing a volume of a substance, such as an aromatic compound 50 within a second chamber housing 31. A second chamber 32 may be positioned anywhere on a second chamber housing 31, but preferably a second chamber 32 may be positioned on a side of a second chamber housing 31 that is opposite to a side that a second contact surface 33 may be positioned.

In some embodiments, the device 100 may comprise one or more aromatic compounds 50. In some embodiments, an aromatic compound may be or comprise any material or compound capable of existing in a gaseous or vapor state and, while in this state, being received in olfactory receptors of the nose, thereby to create a sensation of an aroma in the brain. In further embodiments, an aromatic compound may be or comprise any material or compound capable of sublimation, evaporation, air flow induced erosion, or any other method of being insufflated through the nose 200. Thus, “aromatic” is an objective descriptor of a material or chemical compound capable of being insufflated through the nose 200, regardless of whether an aroma is actually being produced at any given time. Example aromatic compounds 50 include essential oils, such as lavender oil, eucalyptus oil, solid substances such as camphor, other plant substances, plant essences, plant extracts, animal substances, animal extracts, synthetic or occurring aromatic scents, naturally occurring or natural aromatic scents, and synthetic chemicals.

In some embodiments, each chamber 22, 32, may be capable of receiving or containing a volume of one or more aromatic compounds 50. In preferred embodiments, the device 100 may comprise an aromatic compound 50 disposed in a first chamber 22 of a first chamber housing 21 and an aromatic compound 50 disposed in a second chamber 32 of a second chamber housing 31. In further embodiments, a chamber 22, 32, may be capable of receiving or containing a volume of approximately 0.001 to 2.0 milliliters and more preferably 0.001 to 1.0 milliliters. In alternative embodiments, an aromatic compound 50 may be molded, fused, applied, or otherwise disposed on or in another element of the device 100 such as in one or more portions of a retainer 11.

In some embodiments, the device 100 may comprise one or more turbulators 41 which may affect a flow of air which may be passing over or around the device 100. One or more turbulators 41 may be positioned anywhere on the device 100 such as on a first chamber housing 21 and/or a second chamber housing 31. A first chamber housing 21 may comprise a first air flow surface 25 into which a first chamber 22 may be formed, and a turbulator 41 may be disposed on the first chamber housing 21 proximate to the first chamber 22. Preferably, a turbulator 41 may be disposed on the first chamber housing 21 proximate to the first chamber 22 generally between the first chamber 22 and the portion of the first chamber housing 21 that is coupled to the retainer 11. Likewise, a second chamber housing 31 may comprise a second air flow surface 35 into which a second chamber 32 may be formed, and a turbulator 41 may be disposed on the second chamber housing 31 proximate to the second chamber 32. Preferably, a turbulator 41 may be disposed on the second chamber housing 31 proximate, such as between 0.01 to 6.0 millimeters, to the second chamber 32 generally between the second chamber 32 and the portion of the second chamber housing 31 that is coupled to the retainer 11.

Generally, a turbulator 41 may comprise or form a protrusion which extends above an air flow surface 25, 35, and therefore above a chamber 22, 32, of a chamber housing 21, 31. In some embodiments, a turbulator 41 may comprise a ramp surface 42 which may direct air up and away from an air flow surface 25, 35, to cause the air to enter an eddy 301 over a chamber 22, 32, thereby increasing or promoting the dispersion of an aromatic compound 50 from the chamber 22, 32, into the air.

In preferred embodiments, a retainer 11 may position a chamber housing 21, 31, at a desired location on the retainer 11 while also positioning the chamber housings 21, 31, a desired distance from each other. A retainer 11 may comprise a first arm 12 and a second arm 13. A first chamber housing 21 may be coupled to the first arm 12, and a second chamber housing 31 may be coupled to the second arm 13. In some embodiments, a first arm 12 and a second arm 13 may be integrally formed or molded together. In further embodiments, a first arm 12 and a second arm 13 may be coupled together to form the retainer 11. Generally, an arm 12, 13, may comprise a length of material which may be shaped or formed to position a first chamber housings 21 a desired distance from a second chamber housing 31. An arm 12, 13, may comprise a length of between 0.2 and 1.0 centimeters, and more preferably 0.5 to 1.0 centimeters. In still further embodiments, the retainer 11 may comprise a first arm 12 and a second arm 13 which may be coupled together and shaped to separate a first contact surface 23 from a second contact surface 33 by a distance of between 0.1 and 1.0 centimeters, and more preferably between 0.1 and 0.5 centimeters.

In some embodiments, a first arm 12 and/or a second arm 13 may be formed from or comprise a flexible material which may enable the arms 12, 13, and therefore a first contact surface 23 and a second contact surface 33, to be resiliently flexed away from and/or towards each other. A flexible material may include a natural and/or synthetic rubber material such as latex rubber, forms of the organic compound isoprene, Polyacrylate Rubber, Ethylene-acrylate Rubber, Polyester Urethane, a flexible plastic such as high-density polyethylene (HDPE), polyvinyl chloride (PVC), polypropylene (PP), Polystyrene (PS), Polycarbonate (PC), low density polyethylene (LDPE), Polyethylene Terephthalate (PET, PETE, PETG), resilient metals and metal alloys, or any other material which is flexible to allow slight deformation and resilient so as to return to its original shape after deformation.

In some embodiments, the retainer 11 may comprise a bridge 14 having a first end to which a first arm 12 may be coupled and a second or opposing end to which a second arm 13 may be coupled. In other embodiments, a first arm 12 and a second arm 13 may be coupled anywhere on a bridge 14. Generally, a bridge 14 may comprise a length of material which may be used to separate and position a first arm 12 and a second arm 13, and therefore a first contact surface 23 and a second contact surface 33, a desired distance away from each other. In preferred embodiments, a bridge 14 may separate a first arm 12 from a second arm 13 a distance of approximately 0.5 to 1.5 centimeters, so as to accommodate or receive the Columella 203 of the nose 200 while a first contact surface 23 and a second contact surface 33 may be separated by a lesser distance of approximately fifty percent less, such as between 0.25 to 0.75 centimeters, so as to frictionally engage a portion of the septum 204 posterior to the Columella 203.

In some embodiments, the bridge 14 may be made from or comprise a resilient material and the arms 12, 13, may be made from a relatively rigid material so that the resilient material of the bridge 14 may enable the first arm 12 and second arm 13 to resiliently flex away from and/or towards each other. In other embodiments, the bridge 14 may be made from or comprise a relatively rigid material and the arms 12, 13, may be made from a resilient material so that the resilient material of the arms 12, 13, may enable the first arm 12 and second arm 13 to resiliently flex away from and/or towards each other. Example relatively rigid materials include steel alloys, aluminum, aluminum alloys, copper alloys, any other type of metal or metal alloy, various types of hard plastics, such as polyethylene (PE), polypropylene (PP) and polyvinyl chloride (PVC), polycarbonate, Polyethylene Terephthalate (PET, PETE, PETG), nylon, Poly(methyl methacrylate) (PMMA) also known as acrylic, melamine, hard rubbers, fiberglass, carbon fiber, resins, such as epoxy resin, wood, other plant based materials, or any other material including combinations of materials that are substantially rigid. In preferred embodiments, the bridge 14 and the arms 12, 13, may be made from or comprise a resilient material so that the resilient material of the bridge 14 and arms 12, 13, may enable the first arm 12 and second arm 13 to resiliently flex away from and/or towards each other.

In some embodiments, the first arm 12 and second arm 13 may generally form a U-shape. In other embodiments, the first arm 12 and second arm 13 may generally form a C-shape, a V-shape, a J-shape, or any other shape capable of allowing a first contact surface 23 and a second contact surface 33 to contact and be frictionally retained on a portion of the septum 204 posterior to the Columella 203 while preventing the first arm 12 and second arm 13 from squeezing or exerting uncomfortable pressure on the Columella 203. In preferred embodiments, the first arm 12, second arm 13, and bridge 14 may generally form a U-shape. In other embodiments, the first arm 12, second arm 13, and bridge 14 may generally form a C-shape, a V-shape, a J-shape, or any other shape capable of allowing a first contact surface 23 and a second contact surface 33 to contact and be frictionally retained on a portion of the septum 204 posterior to the Columella 203 while preventing the first arm 12, second arm 13, and bridge 14 from squeezing or exerting uncomfortable pressure on the Columella 203. It should be understood to one of ordinary skill in the art that the retainer 11 and the elements of the retainer 11, chamber housings 21, 31, chambers 22, 32, and any other element described herein may be configured in a plurality of sizes and shapes including “T” shaped, “X” shaped, square shaped, rectangular shaped, cylinder shaped, cuboid shaped, hexagonal prism shaped, triangular prism shaped, or any other geometric or non-geometric shape, including combinations of shapes. It is not intended herein to mention all the possible alternatives, equivalent forms or ramifications of the invention. It is understood that the terms and proposed shapes used herein are merely descriptive, rather than limiting, and that various changes, such as to size and shape, may be made without departing from the spirit or scope of the invention.

In some embodiments, the retainer 11 may comprise one or more flexures 15 which may facilitate the ability of the first arm 12 and second arm 13 to resiliently flex away from each other. In further embodiments, a flexure 15 may comprise a recessed, relatively flexible area between two relatively more rigid components, such as a portion of thinner plastic between thicker plastic portions, which may facilitate the ability of the two relatively more rigid components to resiliently flex away from each other. A flexure 15 may be positioned anywhere on the retainer 11. In preferred embodiments, a flexure 15 may be positioned on or proximate to the junction of a first arm 12 and the bridge 14 and another flexure 15 may be positioned on or proximate to the junction of a second arm 13 and the bridge 14. In alternative embodiments, a first arm 12 may be coupled to a second arm 13 and a flexure 15 may be positioned on or proximate to the junction of the first arm 12 and the second arm 13.

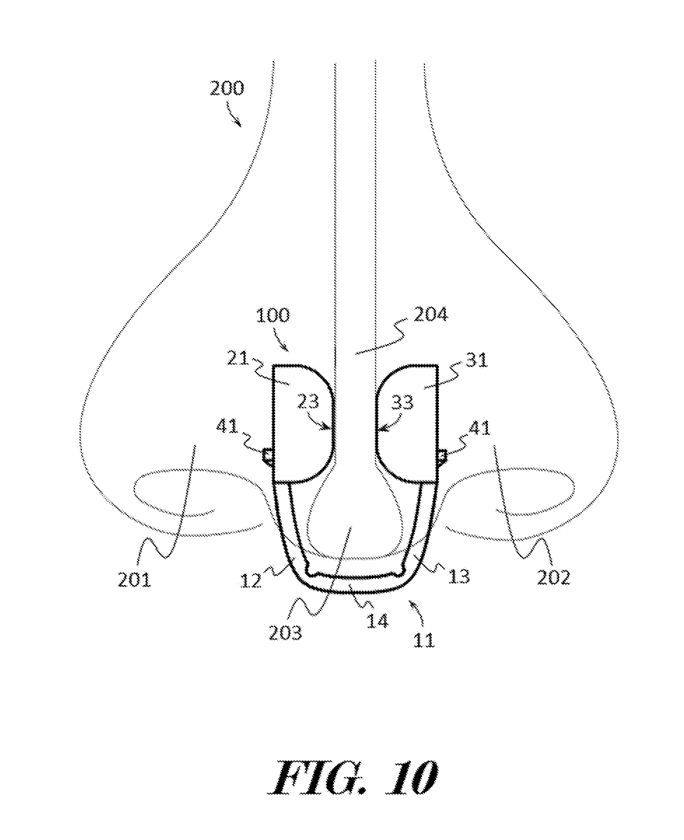

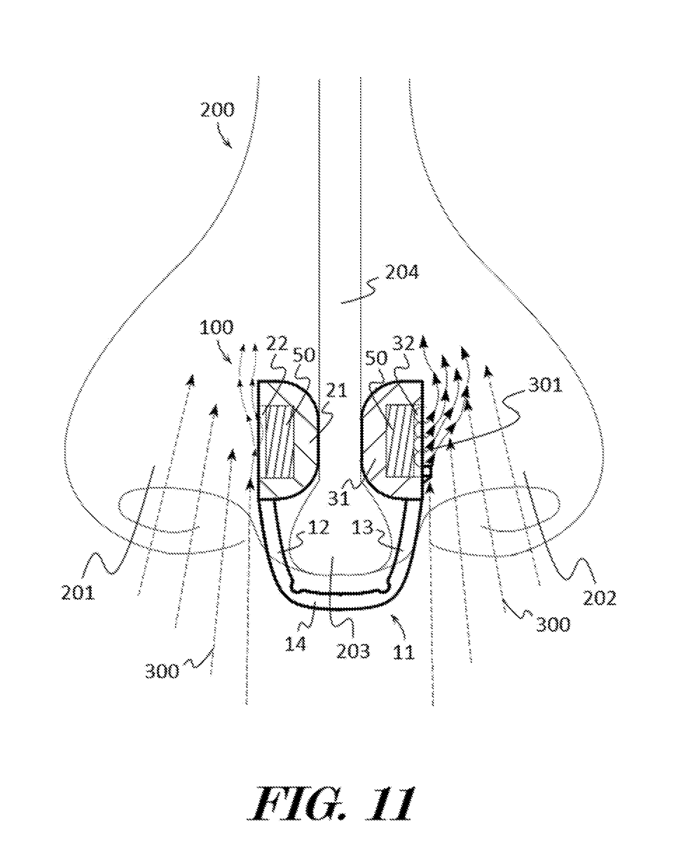

As perhaps best shown in FIGS. 10 and 11, all or portions of the device 100 may inserted or positioned within the nose 200 of a user. Preferably, the device 100 may be configured for use with a nose 200 having a septum 204 which separates a first nostril 201 from a second nostril 202. In some embodiments, the chamber housings 21, 31, may be positioned within the nostrils 201, 202, of the nose 200 so that a portion of the septum 204 may be gripped or frictionally retained between a first contact surface 23 of the first chamber housing 21 and a second contact surface 33 of a second chamber housing 31. In this manner, one or more chambers 22, 32, in the chamber housings 21, 31, may be positioned within one or both of the nostrils 201, 202. In some embodiments, each chamber 22, 32, may contain one or more aromatic compounds 50. As an air flow 300 moves across the chambers 22, 32, the one or more aromatic compounds 50 may be diffused or otherwise picked up by the air flow 300 to be insufflated through the nose 200.

In preferred embodiments, the device 100 may comprise one or more turbulators 41 which may affect the air flow 300 passing over or around the device 100. One or more turbulators 41 may be positioned anywhere on the device 100 such as on a first chamber housing 21 and/or a second chamber housing 31. Each turbulator 41 may comprise a ramp surface 42 which may direct air flow 300 up and away from an air flow surface 25, 35, to cause the air to eddy 301 or otherwise become turbulent over a chamber 22, 32, thereby increasing or promoting the dispersion of an aromatic compound 50 from the chamber 22, 32, into the air flow 300.

While some materials have been provided, in other embodiments, the elements that comprise the device 100 such as the retainer 11, chamber housings 21, 31, contact surfaces 23, 33, optional cushions 24, 34, optional turbulator 41, and/or any other element discussed herein may be made from durable materials such as aluminum, steel, other metals and metal alloys, wood, hard rubbers, hard plastics, fiber reinforced plastics, carbon fiber, fiber glass, resins, polymers or any other suitable materials including combinations of materials. Additionally, one or more elements may be made from or comprise durable and slightly flexible materials such as soft plastics, silicone, soft rubbers, or any other suitable materials including combinations of materials. In some embodiments, one or more of the elements that comprise the device 100 may be coupled or connected together with heat bonding, chemical bonding, adhesives, clasp type fasteners, clip type fasteners, rivet type fasteners, threaded type fasteners, other types of fasteners, or any other suitable joining method. In other embodiments, one or more of the elements that comprise the device 100 may be coupled or removably connected by being press fit or snap fit together, by one or more fasteners such as hook and loop type or Velcro® fasteners, magnetic type fasteners, threaded type fasteners, sealable tongue and groove fasteners, snap fasteners, clip type fasteners, clasp type fasteners, ratchet type fasteners, a push-to-lock type connection method, a turn-to-lock type connection method, a slide-to-lock type connection method or any other suitable temporary connection method as one reasonably skilled in the art could envision to serve the same function. In further embodiments, one or more of the elements that comprise the device 100 may be coupled by being one of connected to and integrally formed with another element of the device 100.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. An intra-nasal clip device, the device comprising:
a. a retainer having a first arm and a second arm;
b. a first chamber housing coupled to the first arm, the first chamber housing comprising a first chamber and a first contact surface, wherein the first chamber and first contact surface are positioned on opposing sides of the first chamber housing; and
c. a second chamber housing coupled to the second arm, the second chamber housing comprising a second chamber and a second contact surface, wherein the second chamber and second contact surface are positioned on opposing sides of the second chamber housing, and wherein the first contact surface and second contact surface are orientated towards each other.

2. The device of claim 1, wherein the first arm and second arm are coupled together with a bridge.

3. The device of claim 2, wherein the first arm, second arm, and bridge generally form a U-shape.

4. The device of claim 1, wherein the retainer comprises a flexure.

5. The device of claim 1, wherein the first arm and second arm are configured to resiliently flex away from each other.

6. The device of claim 1, wherein the retainer comprises a resilient material.

7. The device of claim 1, wherein the device comprises a turbulator.

8. The device of claim 1, wherein the device comprises a first turbulator disposed on the first chamber housing proximate to the first chamber, and wherein the device comprises a second turbulator disposed on the second chamber housing proximate to the second chamber.

9. The device of claim 1, wherein the first chamber housing comprises a resilient material which forms the first contact surface, and wherein the second chamber housing comprises a resilient material which forms the second contact surface.

10. The device of claim 1, wherein the device comprises an aromatic compound disposed in the first chamber and in the second chamber.

11. An intra-nasal clip device for use with a nose having a septum which separates a first nostril from a second nostril, the device comprising:
a. a retainer having a first arm and a second arm;
b. a first chamber housing coupled to the first arm, the first chamber housing comprising a first chamber and a first contact surface, wherein the first chamber and first contact surface are positioned on opposing sides of the first chamber housing, and wherein the first contact surface is configured to contact the septum within the first nostril; and
c. a second chamber housing coupled to the second arm, the second chamber housing comprising a second chamber and a second contact surface, wherein the second chamber and second contact surface are positioned on opposing sides of the second chamber housing, wherein the first contact surface and second contact surface are orientated towards each other, and wherein the second contact surface is configured to contact the septum within the second nostril.

12. The device of claim 11, wherein the first chamber and the second chamber are exposed to an air flow within the nostrils.

13. The device of claim 11, wherein the first arm and second arm are coupled together with a bridge.

14. The device of claim 13, wherein the first arm, second arm, and bridge generally form a U-shape.

15. The device of claim 11, wherein the first arm and second arm are configured to resiliently flex away from each other.

16. The device of claim 11, wherein the retainer comprises a resilient material.

17. The device of claim 11, wherein the device comprises a turbulator.

18. The device of claim 11, wherein the device comprises a first turbulator disposed on the first chamber housing proximate to the first chamber, and wherein the device comprises a second turbulator disposed on the second chamber housing proximate to the second chamber.

19. The device of claim 11, wherein the first chamber housing comprises a resilient material which forms the first contact surface, and wherein the second chamber housing comprises a resilient material which forms the second contact surface.

20. The device of claim 11, wherein the device comprises an aromatic compound.
